SEQUENCE LISTING (75 sequences)

SEQ ID NO: 1 - DNA, 1173 bp, synthetic construct
  source 1..1173: mol_type = other DNA
gttttagctcgcgagattccgccatggatgtcgcttctgctcggggggtctcctcacatcctccatattatagcaaacccatttgttcatcacagtcatcgttgattcggattccgatcagtaaaggatgttgctttgctcgttcttcgaacttgattacttcccttcatgctgcttcgagaggggtgacaaggcgtactagtggtgtacaatggtgttaccgttctattagatttgaccctttcaaagttaatgataagaactcaagaactgtgactgtgagatcggatctttcaggagctgcaacccctgaatctacttatccagaaccagagattaagttgagctcaagactcagagggatatgcttctgtctcgttgctggcatctccgccattgttctcatcgtcctgatgatcattggccatcccttcgtccttctatttgatcgttacaggagaaagttccatcacttcattgctaagctttgggcttccataagcatctacccgttttacaaaacagacatccaaggtttggagaatctgccgtcgtcagacactccttgtgtatacgtttcgaaccaccaaagctttctggatatatacacacttctcagccttggccaaagctataagttcatcagcaagacagggatattcgttattcctgtcatcggttgggctatgtccatgatgggggttgttcccttgaagaggatggacccaagaagccaagtggattgcttaaaacgctgcatggaactagtgaagaagggagcttccgtctttttcttcccagagggaacgaggagtaaggatggtcggttaggtcctttcaagaaaggggcttttacgatagcagctaagacaggagttccagtggtgccaataacgctgatgggaacagggaagatcatgccgacgggtagtgaaggtatactgaatcatggggatgtgagagtgatcatccacaagccgatatatggaagcaaagctgatgttctttgcgaagaggcgagaaacaagatagctgaatctatgaatctcttgagttgaaacgtttgttttttaagcagtgtctctatgaacaatgagaaggctaaaccatttttacatgtcagttttattgtttaaaataaaatttaggcttttcaaaaaaaaaaaaaaaaaa

SEQ ID NO: 2 - DNA, 3255 bp, synthetic construct
  source 1..3255: mol_type = other DNA
atggaggggaacaacacacaccagggcctctcagggggacggggggtaggagaccatcaggacaaacacgtgagtacatggaggggaacaacacacaccagggcctctcagggggacggggggtaggagaccatcaggacaaacacgtgggtacatggaggggaacaacacacaccagggcctctcagcgggacagggggtaggagaccatcaggacaaacacgtggatacatggaggggaacaacacacaccagggcctctcagcgggacagggggtaggagaccatcaggacaaacacgtgggtacatggaggggaacaacacacaccagggcctctcagggggacagggggtaggagaccatcaggacaaacacgtgggtacatggaggggaacaacacacaccagggcctctcagggggacaggggtaggagaccatcaggacaaacacgtgggtacatggagggcaacaacacacaccagggcctctcagggggacagggggtaggagaccatcagtacaaacacgtggatacatggaggggaacagcacacaccagggcctctcagcgggacaggggtaggagaccatcaggacaaacacgtgggtacatggaggggaacaacacacaccagggcctctcagggggacagggggtaggagaccatcaggacaaacacgtgggtacatggaggggaacaacacacaccagggcctctcagggggacggggggtaggagaccatcaggacaaatagctaatgcatgcagggcctcatacctaggtgatgggttgatgggtgcagcaaaccaccatggcacacatttacctatgtatcaaacctatactttctgcacgtgtatcccagaacataaaataaaattaaaaaaatatatacactgattcatgatctcctttctctccttctgaaacactctttaaaactttttagcatttccccctctgtcttccatgtctcctaactacatgtttcttattttccatgtctttattcctgtgttcattttggatagccccttctgacctatattacagtttactagttcactcttcaactgcttctaacatactaatattctgttaaaaccattcatttgggtttaaatttcaattatgttattctctatggacattctatttgttttcttttaatcttcttggccattctctagagtttcctgttccattatgatatttttaattttttgttttactttaaacatactaaatatagttattttattttattttctgtatctgatactttcaataactgcagtctttgctagtcttttttctgtgctcttgctcatagtttttttcgtttgttttcatgattagaaagagagagaagaaggagagtaaagggaggaggaggaggaggagaaaagaagaaagcagagaagaagggacagaaaaaaaggaagttggttctaacgtttctctaacaactggcttcagtgaaacactcccaccttgtggatttttaggttattgaaattaaccagtcttctgggtgcagcacaccaacatggcacatgtatacatatgtaacaaacctgcactttgtgcacatgtaccctaaaacttaaagtacaataaaaaataaaataaaaagctacacaaatttaaaaaaaaagaaatcaacctaattcctagattaccacctcttgattcaaatgctttaaatctaggcttttcatctgagtctttctttttagttattctgtttatcttcaaaacactcctgctttgaatcattcaaaatctacctccctccctctgtttgactaccatcaatttttttgctcattcctaatgcattaatctattagctgtgaatatccaaaaaccctcatttcactgaatctttgacagacccctttgcatcctcttgttcttctaattatttcctcagaaactttatgttctcttttctttacaagcatgtcatagtttatatataatgtgtgtattgtttttatatatacctatatatagcctctttttaaaagcactatacaccatgctttgaaatatattctaaaatcaggtagcatgaaaatggaaacataacatactaaaacatatgggatgcaacaaaagcagttataagagggacatttatagcaataaatgcctacatcaaaaaagaaaaaaaagatctcaaataagcaacctaatattatgcctaaaggagcgagaaaattagagaacaatacaagcccaaagatagcagaaggaaacaaataacaaagatcagagcagaaataatataatagaaactgaaaatttcaataaaaataagaattgttttttgaaaagataaacaaaattaacaaattcttacatagactaagaaaaaagaaaacaaactcagaagtgaaagaagagacattacaactgataccacagaagttaaaaaatcataacatactactataaacaattattcaccagcaaattagataacctagaagaaattgataaactcgtaccaaaactgaatcatgaagaattcaaaatttagaagaaatcatgaataaggaaattaaatcaccaatgaaagttctctcataaaagaaagacccaggattgaatggcttggtggctgaattccaacaaacacttagatgactaacaccaatccttcccaaactcttccaaaaaaaatgaagaagaggaatacttccaaattcatttttcaaaaccagcattaccctgataccaaaaccagagaaggacactataataaaaataaatggcagaccaatactcctgatgaacttggatggaaaaaccttcagccaaatattagcaaatattatttttaaaaaaacacagcaaaaaaattcgccatgcttaagtgggattcatccctgggaagcttattagtcttatttgattcgtgtaatcagaaaatttctatgtctagtgaagagaaatgagagcaatagagactcatagcacctcaacaaatgtccaggcttgagccagttaacaaatacaagtccttcaaatacaaaaaagactgtgaaagaaaatagaacagatcaatgaaactaagaatttgttctttgaaaagataaaactgacaaaccattagctagactagaaaaatgagagaatactcaaagcaataaaatcagaaatgaaagaggaaatattgcaactaataccacagaaatacagaggatcataagaggccactataaacaattacaagccaac

SEQ ID NO: 3 - DNA, 837 bp, synthetic construct
  source 1..837: mol_type = other DNA
atggcctcaggactcccacccccacagcgtggccaggggcttccttggggcccgccaagttcctgggaccctcccaggctagtcagggctcccagcaggagccccctgcaccgagacggccgtgtcccctgctccatgggaccacagcacccactgtcacacacacacagccaccgtcccatcaggcacccacatccccgtggggcccggctcagccaggggttgccagcctcccagggtatccttgacagctgggggaggctggccttggccccatctctctctaccccactgctgggggtcggccctcccacttcctctcctcttcccctaaggtcctgtcacaatgaggagctcccaggggccggcgctgcagccaccctccccgggcaggctggagccggccttccttccacctcgccctccgcttcccacgggtaagctgctcgcaaggcagccggcacgctcgggtcacgaggtcaagggcagcaggagcgaccagggagttgcagtcaccctgccccgtgccacaacctcgttcacaggagggaaatcaggcaggcccagagacggcaagcaacctgtataagatcacacagccagcgcagggtgaggcctgggctctgggcacctgactctcacgtgtcggttgcctctgctcctccctccgtcagtgctggctgccccctgaagctgtcatccagggaccagcaaacagaagtgtctgaatgaggcaacagcatggcccccgccccagcagttaaaggtttgaggcagatccccataggactttgggccaacacctccaaatctctgagcgggacagggagatggtga

SEQ ID NO: 4 - DNA, 975 bp, synthetic construct
  source 1..975: mol_type = other DNA
caaaaaagatctcaaaacagaagaaccaacccaagcagccaaaaccaaagcaacaacttgttgagcagcaccagtagcagccaaagctgcagccaaacctctccaagaaccataacaaacaccagcaaaaaccaaaatagaaaccaaagtaatagccaattgttgaacccacatagatgaagaataagaataagcttgtggaccaggcatagaaaatcttggaacatttttcaaatcttccaaaccataagtagcagcccaacaagaatcccaagtagcttgcaaatcagtaaaaaaagcttcaaaatttggataatcagcagatttcaaaaccttagaaaaagtagtaacacaagttctaccaaaatgaacagattgagatttttcagacaaaacttcatctttacctctagtaacaacaatttgaacaggcaattttctagaatgagcataatgcaacatacctcttttcaatggcaaagaagcaggtttagtagatctatgaccttcaggataaaccaacaaaccaggaacatgagaagaacccaaagtttgatccaaccaagcattaaaagcttctttatcagcaatagtacctctcttaaacaaaacaatacctttcaaaatcatgcaagaagtacaaaaaacagggaaaacaaaataaaccaaccatctagacattaaagcagctctaccttcagtcaaataagcatcaataaaaaaatcagcccaagatctatgattacacaaatacaaacatggaccacctttatacaaagtatgttcaccagcttgcaacaaagtaactctaaaataagcaaccaaagctctagcccaatccaacatatcatttcttttacccaaagaagcgaatctgattctatataaaatagcaaagattggtaaagaccaataaaaaacaaaaacagaaaacaagaaagatggtaaacccaaccatttagtcaaaacagacat

SEQ ID NO: 5 - DNA, 4538 bp, synthetic construct
  source 1..4538: mol_type = other DNA
cttgctgaaaagatgatgttctgaggtattcgtatcgctagcttgatacgcttttaacaaaagtaagcttttcgtttgcaggtttggttacttttctgtacgagatgatatcgctaagtttatagtcatctgtgaaatttctcaaaaacctcatggtttctccatcacccatttttcatttcatttgccgggcggaaaaaaaaaaggaaaaaaaaaaaaaaaaaaaataaatgacacatggaaataagtcaaggattagcggatatgtagttccagtccgggttataccatcacgtgataataaatccaaatgagaatgagggtgtcatatctaatcattatgcacgtcaagattctccgtgactatggctcttttctgaagcatttttcgggcgcccggtggccaaaaactaactccgagcccgggcatgtcccggggttagcgggcccaacaaaggcgcttatctggtgggcttccgtagaagaaaaaaagctgttgagcgagctatttcgggtatcccagccttctctgcagaccgccccagttggcttggctctggtgctgttcgttagcatcacatcgcctgtgacaggcagaggtaataacggcttaaggttctcttcgcatagtcggcagctttctttcggacgttgaacactcaacaaaccttatctagtgcccaaccaggtgtgcttctacgagtcttgctcactcagacacacctatccctattgttacggctatggggatggcacacaaaggtggaaataatagtagttaacaatatatgcagcaaatcatcggctcctggctcatcgagtcttgcaaatcagcatatacatatatatatgggggcagatcttgattcatttattgttctatttccatctttcctacttctgtttccgtttatattttgtattacgtagaatagaacatcatagtaatagatagttgtggtgatcatattataaacagcactaaaacattacaacaaagcaggaaacagctatgaccatgattacgcctaggataacttcgtatagcatacattatacgaagttatgacgacagagaccgggttggcggcgcatttgtgtcccaaaaaacagccccaattgccccaattgaccccaaattgacccattaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgttttagggtaccgactagttccatggcctgtccccacgttgccggtcttgcctcctactacctgtccatcaatgacgaggttctcacccctgcccaggtcgaggctcttattactgagtccaacaccggtgttcttcccaccaccaacctcaagggctctcccaacgctgttgcctacaacggtgttggcatttaggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgttatcaaatctagttaataacttcgtatagcatacattatacgaagttatgctagcgtccggagcggccgcgcatgcaagtcgaccttggcactggccgtcgttttaatagcttcaaaatgtttctactccttttttactcttccagattttaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgtatcacattacaataacaaaactgcaactaccataaaaaaaaattgaaaaatcataaattaaaaaaaaaaaaatcaattgaatttttttttttcatgattacgttttgacatttttcctttttttttctcttattacgatttaccttttttatttatttttttcattttagtattttattcttcgttatttatgtatagaaattttcattttcatttagattcagatttggttatcttttttcattatatatcttttgcactaagtttcaacttaagttctattttttattttttttttctgggccctggagcaatagatatgggatggcttactgcatctcttcaaaatttcacagtcatgctcacccttaagttctcaaccttttagtttttttttttttttttttttattttgtatggcatactaactatacaaatatttatatgtacatttatacagtaacctattatttaccgatattcaccggtattttactttaatagatgctggtaataacgaaaaaaattgataactcccaccgacaattgagaaaaaaagcaaaaaaggaaaaaagggaagcacgaattaatacattctgtgtatttatcttttttatcttcagttttcctccaatctatgggcggccattaagtatgtctattattcttggcccttcagtcttctttggatttcaatgtcttgttggattagaagtcatcaccactttcagcaatcaaagccatgatcattctgtacaagtagaagaagaacatcaataagtggaaacctaactttaggaaactctcccttttatgtttgcctaaagttctgaatatttcggtagcatccaaaagttgaactttattgtagatcttgtttagattgtaagctagaactggtaagttcaataaaaatacaaaccagtaaccgttcagtaagaatagtaatgacaaagcaccatgcaaggcggcttcaggggtaattaacttgttaactt

SEQ ID NO: 6 - DNA, 653 bp, synthetic construct
  source 1..653: mol_type = other DNA
attaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaa

SEQ ID NO: 7 - DNA, 1263 bp, synthetic construct
  source 1..1263: mol_type = other DNA
agagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatagggtaccgactagttccatggcctgtccccacgttgccggtcttgcctcctactacctgtccatcaatgacgaggttctcacccctgcccaggtat

SEQ ID NO: 8 - DNA, 420 bp, synthetic construct
  source 1..420: mol_type = other DNA
ttgatccaaccaagcattaaaagcttctttatcagcaatagtacctctcttaaacaaaacaatacctttcaaaatcatgcaagaagtacaaaaaacagggaaaacaaaataaaccaaccatctagacattaaagcagctctaccttcagtcaaataagcatcaataaaaaaatcagcccaagatctatgattacacaaatacaaacatggaccacctttatacaaagtatgttcaccagcttgcaacaaagtaactctaaaataagcaaccaaagctctagcccaatccaacatatcatttcttttacccaaagaagcgaatctgattctatataaaatagcaaagattggtaaagaccaataaaaaacaaaaacagaaaacaagaaagatggtaaacccaaccatttagtcaaaacaga

SEQ ID NO: 9 - DNA, 168 bp, synthetic construct
  source 1..168: mol_type = other DNA
gcaaatactggctgtttatcaccaggtgcatttttatcaaaggttccagaatttttccagactgcgaatgaaaaacatataactgtgcgtctaacggccaaaagactcatagaacacgatcctgtggaagggaatcttgaatttgattctaccaaccatcctgactat

SEQ ID NO: 10 - DNA, 195 bp, synthetic construct
  source 1..195: mol_type = other DNA
ttttataattttcttcaacatatttttgtttaagccggcattgttgtttttcttcttgtcattcattaaatgagagttgtaaattgaacttgaattaccactactactacttccattggaattagaaaccagtagcttcatcttttctgatatttcctttattagtaagctaaccgccacatcgacatttaaatc

SEQ ID NO: 11 - DNA, 95 bp, synthetic construct
  source 1..95: mol_type = other DNA
tatctacctctgaaaaccaaagtaaaggtagtggtacattggttgtcatattggccattttaatgctaggtgttgcttattatttgttgaacgaa

SEQ ID NO: 12 - DNA, 129 bp, synthetic construct
  source 1..129: mol_type = other DNA
agagatggtgtctcttttcgttagaaaattgtccttcgcgtaattgaaaagcatggaatgcaaattgtgcttataggagaactgtaaaatgtaagaaagcagcacaataaggatgaagttggtgaaaaa

SEQ ID NO: 13 - DNA, 393 bp, synthetic construct
  source 1..393: mol_type = other DNA
tcagaacaagcagcacaacaagcagttaataatgcgggctggtcagttatttcagcagcacaactgggctatgcgggcaaaacagatgcaagaggcacatattatggcgaaacagcgggctatacaacagcacaagcagaagttctgggcaaatatgattcagaaggcaatctgacagcaattggcatttcatttagaggcacaagcggcccgagagaatcactgattggcgatacaattggcgatgttattaatgatctgctggcgggcttcggcccgaaaggctatgcagatggctatacactgaaagcatttggccaactgctgggcgatgttgcaaaatttgcacaagcacatggcctgagcggcgaagatgttgtggttagcggccat

SEQ ID NO: 14 - DNA, 431 bp, synthetic construct
  source 1..431: mol_type = other DNA
attcactgggcggcctggcagttaattcaatggcagcacaatcagatgcaaattggggcggcttttatgcacaatcaaattatgttgcatttgcatcaccgacacaatatgaagcgggcggcaaagttattaatattggctatgaaaatgatccggtttttagagcactggatggcacaacactgacgggcgcatcactgggcgttcatgatgcaccgcatgcatcagcaacaaataatattgttaattttaatgatcattatgcatcagatgcatggaatctgctgccgttttcaattctgaatattccgacatggctgtcacatctgccgtttttttatcaagatggcctgatgagagttctgaattcagagttctattcactgacagataaagattcaacaattattgtgtcaaatctgtcaaatgtt

SEQ ID NO: 15 - DNA, 912 bp, synthetic construct
  source 1..912: mol_type = other DNA
atgagtgtgataggtaggttcttgtattacttgaggtccgtgttggtcgtactggcgcttgcaggctgtggcttttacggtgtaatcgcctctatcctttgcacgttaatcggtaagcaacatttggctcagtggattactgcgcgttgtttttaccatgtcatgaaattgatgcttggccttgacgtcaaggtcgttggcgaggagaatttggccaagaagccatatattatgattgccaatcaccaatccaccttggatatcttcatgttaggtaggattttcccccctggttgcacagttactgccaagaagtctttgaaatacgtcccctttctgggttggttcatggctttgagtggtacatatttcttagacagatctaaaaggcaagaagccattgacaccttgaataaaggtttagaaaatgttaagaaaaacaagcgtgctctatgggtttttcctgagggtaccaggtcttacacgagtgagctgacaatgttgcctttcaagaagggtgctttccatttggcacaacagggtaagatccccattgttccagtggttgtttccaataccagtactttagtaagtcctaaatatggggtcttcaacagaggctgtatgattgttagaattttaaaacctatttcaaccgagaacttaacaaaggacaaaattggtgaatttgctgaaaaagttagagatcaaatggttgacactttgaaggagattggctactctcccgccatcaacgatacaaccctcccaccacaagctattgagtatgccgctcttcaacatgacaagaaagtgaacaagaaaatcaagaatgagcctgtgccttctgtcagcattagcaacgatgtcaatacccataacgaaggttcatctgtaaaaaagatgcattaa

SEQ ID NO: 16 - DNA, 1860 bp, synthetic construct
  source 1..1860: mol_type = other DNA
atgtacaatcctgtggacgctgttttaacaaagataattaccaactatgggattgatagttttacactgcgatatgctatctgcttattgggatcgttcccactgaatgctattttgaagagaattcccgagaagcgtataggtttaaaatgttgttttatcatttctatgtcgatgttttacttattcggtgtgctgaatctagtaagtggattcaggaccctgtttattagtaccatgtttacttacttgatctcaagattttaccgttccaagtttatgccacacttgaatttcatgtttgttatgggtcatttggcaataaatcatatacacgcccaattccttaacgaacagactcaaactaccgttgacattacaagttcacaaatggttttagccatgaaactaacttcttttgcatggtcgtactatgatggttcatgcactagcgaaagcgatttcaaagatttgactgagcatcaaaaatctcgtgctgtcagaggtcatccacccttattaaagttcctggcatatgcatttttctattcaacgttgctaactggcccaagtttcgattatgccgattttgacagctggttgaattgtgagatgttccgtgacttgcctgaaagcaaaaagcctatgagaagacaccaccctggtgaaagaagacagattccaaagaatggtaaacttgcattatggaaagttgttcaaggtcttgcttggatgattttaagtacactaggaatgaagcacttccccgtaaaatacgttttggacaaagatggcttcccaacgagatcttttatattcagaatccattacttattcttgcttggtttcatccatagattcaagtactacgctgcctggactatttcggaaggatcttgtattttgtgcggtttgggttataatggttatgattcaaagacacaaaagatcagatgggatcgtgtcagaaatattgacatttggaccgtagaaacggcgcagaatacgcgtgaaatgttggaagcatggaatatgaatactaacaagtggctaaaatactctgtttatttacgtgtcacaaagaagggcaaaaaacctggtttccgctcaactttgtttactttcctaacttccgcattttggcatggtaccagacctgggtactatctgacttttgcgacaggggctttgtaccaaacatgtggtaaaatctacagacgcaattttagaccaattttcttgcgagaagatggtgtcactcctttgccttctaaaaaaatctacgatttagttggcatatatgcaattaaactagcatttggttacatggtgcaaccatttattatccttgatttgaagccatctttaatggtatggggctctgtttatttctatgttcatattattgttgctttctcatttttcctattcagaggaccatatgctaaacaagttactgaattttttaaatccaaacaacctaaagaaatattcattagaaaacaaaagaagttggaaaaagatatttctgcaagctctccaaacttgggtggtatattgaaggcaaagattgaacatgaaaagggaaagacagcagaagaagaagaaatgaacttaggtattccaccaattgagttagaaaagtgggacaatgctaaggaagattgggaagatttctgcaaagattacaaagaatggagaaataaaaatggtcttgaaatagaagaggaaaacctttctaaagcttttgaaagattcaagcaggaattttctaacgctgcaagtggatcaggtgaacgtgtgagaaaaatgagttttagtggttactcaccaaagcctatttcaaaaaaggaagagtag

SEQ ID NO: 17 - DNA, 903 bp, synthetic construct
  source 1..903: mol_type = other DNA
atggaaaagtacaccaattggagagacaatggtacgggaatagctccatttctaccaaacacaatcaggaaacctagtaaggtgatgacagcgtgtttgttgggtatcctaggggtgaaaaccattataatgctaccattgattatgctgtaccttctaactggccagaacaacttactgggtttgatattgaagtttacattcagttggaaagaggaaattaccgtgcaaggaatcaagaaacgtgacgtaaggaaatccaagcattatccacagaagggcaagctttatatttgcaattgtacctcacctttagatgctttttcagtggtgttattagctcaagggcctgttacgttgttggtcccatccaatgacattgtatacaaagtttccataagagaattcatcaacttcatcctcgccggtgggttagatataaaactctatggccacgaggtagcagagctatctcaattgggcaataccgtgaattttatgtttgctgagggtacctcatgtaatggtaaaagcgtcttaccgtttagtataaccgggaaaaaacttaaagaattcatagacccttcaataaccacaatgaaccccgcaatggccaaaactaaaaaatttgaattgcagaccatccaaatcaaaactaataaaactgccatcaccacattgcccatctccaatatggagtatttatctagatttctgaacaagggcattaatgttaaatgcaagatcaacgagccacaagtactctcggataatttagaggaattacgcgttgcattaaacggtggcgacaaatataaactagtctcacggaagttagatgttgaatctaagaggaattttgtgaaggaatatatcagcgatcaacgtaaaaagaggaagtag

SEQ ID NO: 18 - DNA, 1929 bp, synthetic construct
  source 1..1929: mol_type = other DNA
atgaaggaaacggcgcaggaatacaaggtgtctgctgtaataccgacccttttgaaaaactggatactgcgtgtagtgtatgccacattggaccatatacctccttttgtgtgggaaattctacatgtcatcaccgatatttatttcttctgggttcaaaagctgattaactatgtgcggccacactcaagagtcatttattataacgctattaagaagttagatgagtgtgatacgtatcaaatgtggtgtcagcaagcgtccgtagtggatgaaataacaggcgcaaatttatggcggcggaatttcttttcgaggaggtacgacttcaattctgtcattgaacaatatagcatattggaaaatatgttaagggaggagaagtatgatgtagttaaggagaagttttcgactacaggcccttgtatgttgaggaactttgcgggtattggagataaaaagttgttcacaaaatctttgatgggtacaaagctacttattgaacaatatttgacccgaattctagaaggcttggatatattaaataatcaaactttaactccaacatcctttttccaaagatgtaagttgtcactgggcacaacagcattaatcttgcaaggtggttcattgtttgggctgtttcaccttggggtgattagaggtttattgttacaggacctgatgcctaatattataagtggtagctctatgggggcatgcgttgcaagtttattcggttgcttgtccaatgagcaactaaagcaattattgaccgatgacaacctcttgaatatcatcaagaatgatgtagatttattaaaaagctgtggatacggtaatttagagcagcacctaaacctggggactttgattcagaacctaatacaccacggttattctcaagacgtttatctttttatccggtttgttatgaaatatattgttaaggaaaaaacttttgaagaagtttatcaaattactggaaaagttttcaatattgttattcatccaactgataaatcatgccctaatttactaaattatgtgacaaccccaaacgtattaatcaagtcagccattgaatgctcacttggttctggtgtaatttcagaggatacatcactattatgtaaaaatttagaaaatgaaattgagccttttcttaacatcaataaaaataaacaagtcaaatttcttaccccagaaaatgccaacaatccgagcataacggagagcccctatactaggttaacggaactattcaatgttaataacttcattgtttctttggcgagaccatacttggcaccattggtggtaaatgatttgaagcatgaaataaaaacgtcaaaatattactattataagcattatccaaatatgcctcctataaacgcgaatacagtaagaaaaacacagagatcctcttcgcaatcacccataaaggcaggaactgtcgaggatcttgagccagagcctttaatgagccctgtgcccccaagttcggcagtcaacgattcagcagagtacataatcccagaattgggcataccgcagttgaactttactgaaatggaaccgctggcgtttaaatttaagtatcatctggaaagaaaattaaaaaatattgccactatggaatttcgacataggatggaggtattggataatttaggcctgttgtgctcattaataaaaagactaataattgatgaaaagactcccagatcagctacggagattgctgtggtaccaaggatgaaaagtctatcattaacaaggattattgaaggtcaactaaacaacattccatactggataaaatctggtgaacgaagtacatggcccgcactagctttaattaagacaagatgtgcagtcgaatttaaattagacgacataataagagcaagacggagtaggtag

SEQ ID NO: 19 - DNA, 2733 bp, synthetic construct
  source 1..2733: mol_type = other DNA
atgagcagcaaaatatcagatcttacatctacacaaaataagcccctccttgttacgcaacaactcatcgaaaaatattacgaacagatcctgggcacttcccagaacataattcctattttaaatccgaagaacaagtttattaggcccagtaaggataattcagatgttgaaagggtggaggaggatgctggtaaaagactgcaaactggcaagaacaaaactacgaacaaggtaaatttcaacctggatactggaaacgaggataaacttgacgatgaccaagagacagtaacagaaaatgaaaataatgatatcgagatggttgagacagacgaaggcgaagatgaaaggcaagggtcatctttagccagtaaatgcaaatcatttctttacaacgtttttgtgggaaactatgaaagagacattcttattgacaaagtctgttcacaaaagcaacatgcgatgtcatttgaagaatggtgttctgcgggcgccagattggatgacctcactgggaaaacagaatggaagcagaaattggaaagtcccttgtatgattacaagctaataaaagatttaacatctagaatgcgtgaggagcgcttgaataggaattacgctcaattgttgtacatcattaggacgaattgggtacgaaacctgggaaatatggggaatgtaaacctgtataggcactctcatgtaggcaccaaatatttaattgacgagtatatgatggagtctaggttagcgctagaatctttaatggagtctgatcttgatgatagttaccttttgggtatactgcaacaaacgagaagaaatattggtcgtaccgctttagttctcagtgggggtggaacttttggtcttttccacatcggtgtccttggtactctatttgaattggatttattacccagagtgattagtggtagcagtgctggtgcaattgtagccagcatattatctgtccatcacaaagaagaaattccggttttactaaatcatattttggataaagaattcaacattttcaaagacgataaacagaaaagtgaaagcgagaatttgttaataaaaatatctaggttcttcaaaaacggtacgtggtttgataacaagcatctggtaaatacaatgatagaatttttgggagatttgacatttagggaagcttacaatagaacgggtaaaattttgaatataaccgtttcgccggcatctttatttgaacaaccgcgcttgctgaataatttgactgcaccaaacgtcctgatttggtccgccgtatgtgcatcatgttcactaccgggaattttcccctcgagcccactttacgaaaaagatccaaaaacgggagaaaggaaaccatggactggtagtagttcggtcaaatttgtcgatggttctgtggacaatgacttgcccatttctcgtctttctgaaatgttcaatgtggaccatattatcgcatgccaggtgaatattcacgtatttccctttttgaaactatcactatcctgtgttggcggggaaattgaggacgaatttagtgcaagattaaagcaaaacttatcaagtatatacaattttatggccaatgaagctattcatattctagaaattggaagtgagatgggaattgccaaaaacgcgcttacaaaactgagatcggtattatctcaacaatattctggtgacatcactattttgcccgacatgtgtatgctttttagaataaaggagctgttgtcaaacccaacaaaagaatttttattaagggaaatcaccaatggtgcaaaagctacgtggcccaaggtttccattattcaaaatcactgtggccaggaatttgctctggataaggcgatttcttatatcaaaggtaggatgattgtcacctcctctttaaaaacccccttccaatttgctgattcagtcattggattaattaaagctccagagcaaacgtcagatgagtccaaaaacccagaaaattcaacattgctaactaggactccaaccaagggtgacaatcatatttccaatgttttagatgacaacttattagaatcagaatcgacaaactctttgctattgttacgtgagaatgcaagcacatatgggcggtcaccttccgggtttagaccgcggtattccattacgtccgcttccctcaatccgcgtcaccaaagaaggaaatcagatactatttcaacttcaaggcgaccagccaaatccttttcattttcagttgcttctcccacatcaaggatgttgaggcaatccagcaaaatcaatggacacccaccaccaattctgcagaaaaaaacaagtatgggccggctaatgtttcctatggatgccaagacctatgacccggaaagccatgaacttatcccacattctgccagcattgaaacacctgccatggtagacaagaaattgcattttggccgaaagagtagatacttgaggcatatgaacaaaaaatgggtcagcagtagcaacatattatacacagattcggataaagaagaccatcctacattgagactgataagtaacttcgattcagacgcaatgattcatagtgatttagcgggcaatttcaggcgtcatagcattgatggaagacccccttctcaagctacaaagagctcaccgtttcgatcgaggccttcttcttcaacgcagcacaaaagcaccaccagttttactcaataa

SEQ ID NO: 20 - DNA, 2250 bp, synthetic construct
  source 1..2250: mol_type = other DNA
atgtctaataccttgccagtaacagaatttcttttatccaaatactacgaactttccaacactcctgccacagactcatcatcgctcttcaaatggttataccacaagactctctcccgtaaacagctgctcatatccgatttgtcttcccagaaaaaacacgccatctcctacgatcaatggaatgacatagcgtcgcgattagacgacttaacgggactttccgaatggaaaacaatcgacgagtcctcgttgtataactataagctgctgcaagacttgaccatccgtatgcgccatttgagaaccactcacgactaccatcgcctgctttatctgattagaactaagtgggtccgtaatcttggcaacatgaataacgttaatctttacaggcactcgcataccggcacaaaacaaattatacatgactacctagaagagtctcaagcagtgctcaccgcgctgatccatcagtcaaatatgaacgatcactatctcctcggtatcttgcaacagacgagaagaaacatagggcgcacggctctcgtgcttagcggtgggagcactttcggccttttccacattggtgttcttgccgccctttttgaatcggacctgatgcctaaggtgatcagcggtagcagtgctggcgccattgttgccagcatattttgcgtccacacgacccaggaaattccctccttgttgaccaatgtacttaatatggagtttaacatcttcaatgacgacaattccaaatctcccaacgaaaacttactaatcaagatatcgaggttctgccaaaacggtacctggttcaataatcaacctttgattaacacaatgctttcgtttttaggaaacttgacctttagggaagcctacaacaagactggaaaaatcctgaatatcacagtctcgcctgcttccatatacgaacagccgaaactactaaacaatttaaccgctccaaacgttctcatctggtctgccgtatgcgcttcttgttctctacctggagttttcccctccacgccgctattcgagaaagatcctcacactggaaagattaaagagtggggggcaacgaatttacatttatcaaacatgaaattcatggacggatctgtagataatgacatgcccatttctcgtctttctgaaatgttcaatgtcgaccacattatcgcctgccaggtaaatatacatgtctttcccctgttaaagttttcaaacacttgcgttgggggtgaaattgaaaaggaaattaccgcccgtttcagaaaccaagtaacaaagatcttcaaatttttttccgacgaaactattcattttttagacatcctaaaggagcttgagttccatccctatttgatgaccaaattgaaacacctttttttacaacaatactccggcaatgtcacaattttacccgatctatcaatggttggtcaattccacgaagtattgaagaacccgtctcaacttttcctattgcaccaaaccactttaggtgcaagagctacctggccgaaaatttccatgattcaaaataattgtggccaagaattcgctctggataaggccatcacattcctaaaggagaaaataataatctcttcgtcaataaaaaaccctttacaattctaccaacctcgattcagtgagcaaatcaaatctctttccataatggatgctgacttgccgggagttgacttggaagaatcctcctccaattcactatcaattatcaagtctcccaacaaaacagcggcaccgggaagatttcctcttcagccattgccttctccatcttctaccttcaacaagaggaaaatggatatgttatcgccttctccatcgccttctacatctccacaacgttcaaaatcttcattcacgcagcagggtacaaggcagaaggcaaattctttatcgtttgccatcggtgcatctagtttacggctaaagaaatcaccattgaaggttccatcacgacctcaattcaaaaaaagatcttcttattataatcaaaatatgtcggcagagatgaggaaaaatagaaaaaaatctggaacaatttcctcttatgatgttcaaacaaactcggaagattttcccataccggctattgaaaacggttcatttgataacactttattcaatccaagcaggttccccatggacgctatgtcggctgccacaaacgacaatttcatgaacaattcagacatttttcaaaattga

SEQ ID NO: 21 - DNA, 311 bp, synthetic construct
  source 1..311: mol_type = other DNA
tctgttttgactaaatggttgggtttaccatctttcttgttttctgtttttgttttttattggtctttaccaatctttgctattttatatagaatcagattcgcttctttgggtaaaagaaatgatatgttggattgggctagagctttggttgcttattttagagttactttgttgcaagctggtgaacatactttgtataaaggtggtccatgtttgtatttgtgtaatcatagatcttgggctgatttttttattgatgcttatttgactgaaggtagagctgctttaatgtctagatggttggttat

SEQ ID NO: 22 - DNA, 33 bp, unidentified
  source 1..33: mol_type = other DNA
agtattatctctgctggtcggtacttaaattgg

SEQ ID NO: 23 - DNA, 61 bp, unidentified
  source 1..61: mol_type = other DNA
aaaaaatcggatgttgaatgggcataaatataaatgtatatataagctattacgccagctg

SEQ ID NO: 24 - DNA, 37 bp, unidentified
  source 1..37: mol_type = other DNA
tcatgacattgacgagagtttctctcgtacccatatt

SEQ ID NO: 25 - DNA, 46 bp, unidentified
  source 1..46: mol_type = other DNA
tcaggggtaattaacttgttaactttggagaagtaaatgaaaaatg

SEQ ID NO: 26 - DNA, 66 bp, unidentified
  source 1..66: mol_type = other DNA
gaaagatggtaaacccaaccatttagtcaaaacagacatatagcttcaaaatgtttctactccttt

SEQ ID NO: 27 - DNA, 61 bp, unidentified
  source 1..61: mol_type = other DNA
cgaatacctcagaacatcatcttttcagcaagtagttctagaaaacttagattagattgct

SEQ ID NO: 28 - DNA, 73 bp, unidentified
  source 1..73: mol_type = other DNA
cataaatataaatgtatatataagctattacgccagctgaattggagcgacctcatgctatactgagaaagca

SEQ ID NO: 29 - DNA, 55 bp, unidentified
  source 1..55: mol_type = other DNA
cgaagatgttgtggttagcggccataattcttcgccagaggtttggtcaagtctc

SEQ ID NO: 30 - DNA, 48 bp, unidentified
  source 1..48: mol_type = other DNA
tgcttgttgtgctgcttgttctgacaaaaaagatctcaaaacagaaga

SEQ ID NO: 31 - DNA, 68 bp, unidentified
  source 1..68: mol_type = other DNA
aaaggagtagaaacattttgaagctatatgtctgttttgactaaatggttgggtttaccatctttctt

SEQ ID NO: 32 - DNA, 60 bp, unidentified
  source 1..60: mol_type = other DNA
gcaatctaatctaagttttctagaactacttgctgaaaagatgatgttctgaggtattcg

SEQ ID NO: 33 - DNA, 74 bp, unidentified
  source 1..74: mol_type = other DNA
tgttttgtatgctatttcatttttcatttacttctccaaagttaacaagttaattacccctgaagccgccttgc

SEQ ID NO: 34 - DNA, 66 bp, unidentified
  source 1..66: mol_type = other DNA
aaccaacaaaccaggaacatgagaagaacccaaagtcatatagcttcaaaatgtttctactccttt

SEQ ID NO: 35 - DNA, 66 bp, unidentified
  source 1..66: mol_type = other DNA
aaaggagtagaaacattttgaagctatatgactttgggttcttctcatgttcctggtttgttggtt

SEQ ID NO: 36 - DNA, 54 bp, unidentified
  source 1..54: mol_type = other DNA
ttgaccaaacctctggcgaagaattatggccgctaaccacaacatcttcgccgc

SEQ ID NO: 37 - DNA, 51 bp, unidentified
  source 1..51: mol_type = other DNA
tcttctgttttgagatcttttttgtcagaacaagcagcacaacaagcagtt

SEQ ID NO: 38 - DNA, 59 bp, unidentified
  source 1..59: mol_type = other DNA
aaaccaggaacatgagaagaacccaaagtatggccgctaaccacaacatcttcgccgct

SEQ ID NO: 39 - DNA, 65 bp, unidentified
  source 1..65: mol_type = other DNA
aaaggagtagaaacattttgaagctatatgtcagaacaagcagcacaacaagcagttaataatgc

SEQ ID NO: 40 - DNA, 38 bp, unidentified
  source 1..38: mol_type = other DNA
ttaccttcatatgacaaaatttcgttggactcatcgtc

SEQ ID NO: 41 - DNA, 60 bp, unidentified
  source 1..60: mol_type = other DNA
tacctcagaacatcatcttttcagcaagtatttttttgctcgaattcactgacggaggga

SEQ ID NO: 42 - DNA, 50 bp, unidentified
  source 1..50: mol_type = other DNA
gcttcaggggtaattaacttgttaacttcacatttttagagtagtatata

SEQ ID NO: 43 - DNA, 39 bp, unidentified
  source 1..39: mol_type = other DNA
ctttagcaatatcggacaaacagccgtattttttattct

SEQ ID NO: 44 - DNA, 39 bp, unidentified
  source 1..39: mol_type = other DNA
ctatcttgggggtacgaaacttagcaatatatttagcaa

SEQ ID NO: 45 - DNA, 51 bp, unidentified
  source 1..51: mol_type = other DNA
tggaccagaactacctgtgaaattaatgttggaagcgatcagcagcagaat

SEQ ID NO: 46 - DNA, 48 bp, unidentified
  source 1..48: mol_type = other DNA
ctaacaaaatagcaaatttcgtcaaaaaatggccaaaaacgacagatg

SEQ ID NO: 47 - DNA, 40 bp, unidentified
  source 1..40: mol_type = other DNA
gtgaagacaaattatctaaggtccaactctacgaagattt

SEQ ID NO: 48 - DNA, 38 bp, unidentified
  source 1..38: mol_type = other DNA
atagacgtttctaaacccaaatgaatccaagttgccga

SEQ ID NO: 49 - DNA, 64 bp, unidentified
  source 1..64: mol_type = other DNA
tcaatgacgaggttctcacccctgcccaggtatcttgtattttgtcacctcgtaggagctactt

SEQ ID NO: 50 - DNA, 45 bp, unidentified
  source 1..45: mol_type = other DNA
tcctagcgctcaccaagctctgaaatacttatgaaggcatgcatg

SEQ ID NO: 51 - DNA, 42 bp, unidentified
  source 1..42: mol_type = other DNA
aggatcattctggtaataccccatttaaaatacctcatcgaa

SEQ ID NO: 52 - DNA, 63 bp, unidentified
  source 1..63: mol_type = other DNA
gtttccctccgtcagtgaattcgagcaaaaaaatacttgctgaaaagatgatgttctgaggta

SEQ ID NO: 53 - DNA, 50 bp, unidentified
  source 1..50: mol_type = other DNA
tatatactactctaaaaatgtgaagttaacaagttaattacccctgaagc

SEQ ID NO: 54 - DNA, 51 bp, unidentified
  source 1..51: mol_type = other DNA
ttctgctgctgatcgcttccaacattaatttcacaggtagttctggtccat

SEQ ID NO: 55 - DNA, 46 bp, unidentified
  source 1..46: mol_type = other DNA
atctgtcgtttttggccattttttgacgaaatttgctattttgtta

SEQ ID NO: 56 - DNA, 48 bp, unidentified
  source 1..48: mol_type = other DNA
catgcatgccttcataagtatttcagagcttggtgagcgctaggagtc

SEQ ID NO: 57 - DNA, 55 bp, unidentified
  source 1..55: mol_type = other DNA
ctacgaggtgacaaaatacaagatacctgggcaggggtgagaacctcgtcattga

SEQ ID NO: 58 - DNA, 42 bp, unidentified
  source 1..42: mol_type = other DNA
atagcctcgacgccagctttgattagttcatccgggttcccg

SEQ ID NO: 59 - DNA, 69 bp, unidentified
  source 1..69: mol_type = other DNA
cttcaggggtaattaacttgttaactttatcgtttccacttttttctgtcttattttttttattgatag

SEQ ID NO: 60 - DNA, 72 bp, unidentified
  source 1..72: mol_type = other DNA
acatctttgagttgccgttaagccttgctgaaaagatgatgttctgaggtattcgtatcgctagcttgatac

SEQ ID NO: 61 - DNA, 54 bp, unidentified
  source 1..54: mol_type = other DNA
atacctcagaacatcatcttttcagcaaggcttaacggcaactcaaagatgtga

SEQ ID NO: 62 - DNA, 42 bp, unidentified
  source 1..42: mol_type = other DNA
ttggtcctattcagcgaccaatcagtgcttcctgcccacttt

SEQ ID NO: 63 - DNA, 55 bp, unidentified
  source 1..55: mol_type = other DNA
aaaagtggaaacgataaagttaacaagttaattacccctgaagccgccttgcatg

SEQ ID NO: 64 - DNA, 37 bp, unidentified
  source 1..37: mol_type = other DNA
gggatcctcttgcgaagcacgctcgctgggcctggaa

SEQ ID NO: 65 - DNA, 64 bp, unidentified
  source 1..64: mol_type = other DNA
acaaaatagcaaatttcgtcaaaaagaaaacacgggcttgcttatatatcctctagatattctt

SEQ ID NO: 66 - DNA, 77 bp, unidentified
  source 1..77: mol_type = other DNA
gagtacaggtatatgtaataaaagtctgaattaatttcacaggtagttctggtccattggtgaaagtttgcggcttg

SEQ ID NO: 67 - DNA, 62 bp, unidentified
  source 1..62: mol_type = other DNA
gaactacctgtgaaattaattcagacttttattacatatacctgtactcccttcaataatta

SEQ ID NO: 68 - DNA, 45 bp, unidentified
  source 1..45: mol_type = other DNA
gttattatgttggcaatggaaaagttgcgtaccgagataccgtaa

SEQ ID NO: 69 - DNA, 62 bp, unidentified
  source 1..62: mol_type = other DNA
atataagcaagcccgtgttttctttttgacgaaatttgctattttgttagagtcttttacac

SEQ ID NO: 70 - DNA, 36 bp, unidentified
  source 1..36: mol_type = other DNA
tgctggagcaagctccgtctccagctcgggaagtgt

SEQ ID NO: 71 - DNA, 66 bp, unidentified
  source 1..66: mol_type = other DNA
cgaggttctcacccctgcccaggtattgaacaattcagacatttttcaaaattgaaatgaaagaca

SEQ ID NO: 72 - DNA, 69 bp, unidentified
  source 1..69: mol_type = other DNA
acaaggaagacggttctgtttcgttgctagagcttggtgagcgctaggagtcactgccaggtatcgttt

SEQ ID NO: 73 - DNA, 61 bp, unidentified
  source 1..61: mol_type = other DNA
cctagcgctcaccaagctctagcaacgaaacagaaccgtcttccttgtgctgtttatgatg

SEQ ID NO: 74 - DNA, 54 bp, unidentified
  source 1..54: mol_type = other DNA
atttcctatgaataatcaagacataatgaattttcaatggctattgaacttccc

SEQ ID NO: 75 - DNA, 69 bp, unidentified
  source 1..69: mol_type = other DNA
aattttgaaaaatgtctgaattgttcaatacctgggcaggggtgagaacctcgtcattgatggacaggt

(19) United States
(12) Patent Application Publication
(10) Pub. No.: US 2024/0067996 A1
(43) Pub. Date: Feb. 29, 2024

(54) SACCHAROMYCES CEREVISIAE STRAIN FOR PRODUCING HUMAN MILK LIPID SUBSTITUTE AND USE THEREOF

(71) Applicants: Jiangnan University, Wuxi (CN); Inner Mongolia Mengniu Dairy (Group) Company Limited, Huhhot (CN)

(72) Inventors: Long LIU, Wuxi (CN); Jian Chen, Wuxi (CN); Xueqin LV, Wuxi (CN); Wenyang Wu, Wuxi (CN); Guocheng Du, Wuxi (CN); Jianghua Li, Wuxi (CN); Guolin Zhou, Wuxi (CN); Yanfeng Liu, Wuxi (CN); Chenyang Zhang, Wuxi (CN)

(21) Appl. No.: 18/499,560

(22) Filed: Nov. 1, 2023

Related U.S. Application Data
(63) Continuation of application No. PCT/CN2022/143077, filed on Dec. 29, 2022.

(30) Foreign Application Priority Data
Aug. 2, 2022 (CN) 202210921790.1

(51) Int. Cl. C12P 7/6409; C12N 9/10; C12N 9/20; C12N 15/81

(52) U.S. Cl. CPC C12P 7/6409; C12N 9/1029; C12N 9/20; C12N 15/81; C12Y 203/01051; C12Y 301/01003; C12N 2800/102

(57) ABSTRACT

The invention provides a Saccharomyces cerevisiae strain for producing a human milk lipid substitute. By integrating a heterologous lysophosphatidic acid acyltransferase into Saccharomyces cerevisiae and knocking out its own natural lysophosphatidic acid acyltransferase, the content of palmitic acid (C16:0) at Sn-2 position of triacylglycerol produced by Saccharomyces cerevisiae is increased, to synthesize a human milk lipid substitute. On this basis, a metabolic pathway related gene is knocked out, to further increase the content of human milk lipid substitute in the product. In the present invention, a human milk lipid substitute is de novo synthesized by Saccharomyces cerevisiae for the first time, in which the total fatty acid is 15% or more, and the relative content of C16:0 at Sn-2 position reaches about 60%.

This application is a Continuation application of PCT/CN2022/143077, filed on Dec. 29, 2022, which claims priority to Chinese Patent Application No. CN 202210921790.1, filed on Aug. 2, 2022, which is incorporated by reference for all purposes as if fully set forth herein.

A Sequence Listing XML file named “10015_0136.xml” created on Nov. 1, 2023, and having a size of 91,425 bytes, is filed concurrently with the specification. The sequence listing contained in the XML file is part of the specification and is herein incorporated by reference in its entirety.

### FIELD OF THE INVENTION

The invention relates to the field of biological technology, and more particularly to a Saccharomyces cerevisiae strain for producing human milk lipid substitute and use thereof.

### DESCRIPTION OF THE RELATED ART

Infants are populations in a special growth stage. In this stage, they need a lot of comprehensive nutrients, and the quantity and quality of nutrient intake in this stage directly affect the future growth and development of infants. Human milk is the most direct source of energy and nutrients for infants, which contains a large number of special ingredients that can promote the healthy growth and development of infants.

Human milk lipid is the main nutrient in human milk, accounting for 3%-5% of human milk. 98% of the human milk lipid is triacylglycerols, which are the main energy source for infant growth and development. At the age of 0-6 months, the human milk lipid provides 40%-50% of the energy needed for infants, and the human milk lipid also provides essential fatty acids for infants. Studies show that both the fatty acid composition and the distribution position of fatty acids in human milk lipids are key factors determining whether infants can absorb nutrients in human milk. Nearly 50% of the fatty acids at position 2 (Sn-2) of human milk lipid is palmitic acid. This particular triacylglycerols structure enables infants to better absorb calcium ions, which can effectively prevent diarrhea, avoid infant malnutrition, and reduce infant mortality. However, although human feeding is the best way to feed infants, there are situations where human feeding cannot be used for some reasons. In such cases, infant formula suitable for infants, with human milk as the gold standard, becomes an ideal product to replace human milk.

In recent years, researchers in China and other countries not only focused on the composition and contents of fatty acids in human milk, but also paid more attention to the location distribution of fatty acids in triacylglycerol of human milk. In human milk lipids, palmitic acid (16:0) is esterified to the middle (Sn-2 or 3) position in the main chain of glycerol, and oleic acid (18:1) is mainly esterified to the outer (Sn-1,3) position, such that triacylglycerols (TGs) has a unique stereoisomeric structure promoting the absorption of nutrients in infants' intestines. However, most of the lipid used in infant formula milk powder is derived from animals. Different from the structure of triacylglycerol in human milk, unsaturated fatty acids such as C18:1 in animal-derived triacylglycerol are mainly located at the middle (Sn-2 or 3) position in the main chain of glycerol, and saturated fatty acids such as C16:0 are mainly located at the outer (Sn-1,3) position in the main chain of glycerol, which is not conducive to the absorption and growth of infants.

At present, the human milk lipid substitute (triacylglycerol with the particular structure) is mainly enzymatically synthesized, which occupies a dominating position in the market. For example, camellia seed oil is used as a raw material, and palmitic acid is used as an acyl donor to enzymatically synthesize triacylglycerol rich in palmitic acid at Sn-2 position in an organic solvent system by Xiong Zhiqin (Enzymatic Preparation and Property Study of Human Milk Lipid Substitute). With the production and development of human milk lipid substitutes, biological de novo synthesis has attracted more and more attention because of its easy control and ability to synthesize various desired products. Industrial production of strains through genetic engineering to produce specific lipid compositions has great potential. Meanwhile, genetic engineering methods are used to construct de novo synthetic products from genetically engineered strains, which have many advantages such as low cost, unrestricted raw materials, simple extraction process, no seasonality, short production time, and low environmental pollution, so it is favored by scholars. For example, an Arabidopsis plant that can synthesize human milk substitutes constructed by genetic engineering in Arabidopsis thaliana. Human milk substitute is also synthesized in microorganisms by researchers by changing the substrate supply. However, there is no report on the preparation of a human milk lipid substitute by de novo synthesis.

### SUMMARY OF THE INVENTION

To solve the above problems, the present invention provides an engineered Saccharomyces cerevisiae strain for synthesizing a human milk lipid substitute. The human milk lipid substitute is synthesized by integrating a heterologous lysophosphatidic acid acyltransferase into Saccharomyces cerevisiae and knocking out its natural lysophosphatidic acid acyltransferase, the content of palmitic acid (16:0) at Sn-2 position of triacylglycerol produced by Saccharomyces cerevisiae is increased. On this basis, a metabolic pathway-related gene is knocked out, to further increase the content of human milk lipid substitute in the product.

The first object of the present invention is to provide a recombinant Saccharomyces cerevisiae strain for producing a human milk lipid substitute. In the recombinant Saccharomyces cerevisiae strain, a lysophosphatidic acid acyltransferase CrlPAAT1 is expressed, SLC1, ALE1, and LOA1 genes encoding lysophosphatidic acid acyltransferase are knocked out, and TGL3, TGL4 and TGL5 genes encoding triglyceride lipase are knocked out.

In the lysophosphatidic acid acyltransferase CrlPAAT1, the self-localization signal peptide is knocked out, and an endoplasmic reticulum localization signal peptide is linked to the C terminal.

Preferably, the lysophosphatidic acid acyltransferase CrlPAAT1 has a nucleotide sequence as shown in SEQ ID NO: 4. Specifically, the sequence is as follows:

CAAAAAAGATCTCAAAACAGAAGAACCAACCCAAGCAGCCAAAACCAAAG
CAACAACTTGTTGAGCAGCACCAGTAGCAGCCAAAGCTGCAGCCAAACCT
CTCCAAGAACCATAACAAACACCAGCAAAAACCAAAATAGAAACCAAAGT
AATAGCCAATTGTTGAACCCACATAGATGAAGAATAAGAATAAGCTTGTG
GACCAGGCATAGAAAATCTTGGAACATTTTTCAAATCTTCCAAACCATAA
GTAGCAGCCCAACAAGAATCCCAAGTAGCTTGCAAATCAGTAAAAAAAGC
TTCAAAATTTGGATAATCAGCAGATTTCAAAACCTTAGAAAAAGTAGTAA
CACAAGTTCTACCAAAATGAACAGATTGAGATTTTTCAGACAAAACTTCA
TCTTTACCTCTAGTAACAACAATTTGAACAGGCAATTTTCTAGAATGAGC
ATAATGCAACATACCTCTTTTCAATGGCAAAGAAGCAGGTTTAGTAGATC
TATGACCTTCAGGATAAACCAACAAACCAGGAACATGAGAAGAACCCAAA
GTTTGATCCAACCAAGCATTAAAAGCTTCTTTATCAGCAATAGTACCTCT
CTTAAACAAAACAATACCTTTCAAAATCATGCAAGAAGTACAAAAAACAG
GGAAAACAAAATAAACCAACCATCTAGACATTAAAGCAGCTCTACCTTCA
GTCAAATAAGCATCAATAAAAAAATCAGCCCAAGATCTATGATTACACAA
ATACAAACATGGACCACCTTTATACAAAGTATGTTCACCAGCTTGCAACA
AAGTAACTCTAAAATAAGCAACCAAAGCTCTAGCCCAATCCAACATATCA
TTTCTTTTACCCAAAGAAGCGAATCTGATTCTATATAAAATAGCAAAGAT
TGGTAAAGACCAATAAAAAACAAAAACAGAAAACAAGAAAGATGGTAAAC
CCAACCATTTAGTCAAAACAGACAT.

Preferably, the self-localization signal peptide of the lysophosphatidic acid acyltransferase CrlPAAT1 has a nucleotide sequence as shown in SEQ ID NO: 8.

Specifically, the sequence is as follows:

TTGATCCAACCAAGCATTAAAAGCTTCTTTATCAGCAATAGTACCTCTCT
TAAACAAAACAATACCTTTCAAAATCATGCAAGAAGTACAAAAAACAGGG
AAAACAAAATAAACCAACCATCTAGACATTAAAGCAGCTCTACCTTCAGT
CAAATAAGCATCAATAAAAAAATCAGCCCAAGATCTATGATTACACAAAT
ACAAACATGGACCACCTTTATACAAAGTATGTTCACCAGCTTGCAACAAA
GTAACTCTAAAATAAGCAACCAAAGCTCTAGCCCAATCCAACATATCATT
TCTTTTACCCAAAGAAGCGAATCTGATTCTATATAAAATAGCAAAGATTG
GTAAAGACCAATAAAAAACAAAAACAGAAAACAAGAAAGATGGTAAACCC
AACCATTTAGTCAAAACAGA.

Preferably, the endoplasmic reticulum localization signal peptide is HDEL, the nucleotide sequence as shown in SEQ ID NO: 13. The endoplasmic reticulum localization signal peptide is expressed in the heterologous lysophosphatidic acid acyltransferase, to modify the heterologous acyltransferase, such that the heterologous acyltransferase can be retained in the endoplasmic reticulum to exert its function. Specifically, the sequence is as follows:

TCAGAACAAGCAGCACAACAAGCAGTTAATAATGCGGGCTGGTCAGTTAT
TTCAGCAGCACAACTGGGCTATGCGGGCAAAACAGATGCAAGAGGCACAT
ATTATGGCGAAACAGCGGGCTATACAACAGCACAAGCAGAAGTTCTGGGC
AAATATGATTCAGAAGGCAATCTGACAGCAATTGGCATTTCATTTAGAGG
CACAAGCGGCCCGAGAGAATCACTGATTGGCGATACAATTGGCGATGTTA
TTAATGATCTGCTGGCGGGCTTCGGCCCGAAAGGCTATGCAGATGGCTAT
ACACTGAAAGCATTTGGCCAACTGCTGGGCGATGTTGCAAAATTTGCACA
AGCACATGGCCTGAGCGGCGAAGATGTTGTGGTTAGCGGCCAT.

Preferably, plasmid pMHyLp-LEU is used as an expression vector for the gene expressing the lysophosphatidic acid acyltransferase CrlPAAT1, and pMHyLp-LEU has a nucleotide sequence as shown in SEQ ID NO: 5.

Preferably, SLC1 has a nucleotide sequence as shown in SEQ ID NO: 15, ALE1 has a nucleotide sequence as shown in SEQ ID NO: 16, and LOA1 has a nucleotide sequence as shown in SEQ ID NO: 17.

Preferably, TGL3 has a nucleotide sequence as shown in SEQ ID NO: 18, TGL4 has a nucleotide sequence as shown in SEQ ID NO: 19, and TGL5 has a nucleotide sequence as shown in SEQ ID NO: 20.

Preferably, the recombinant Saccharomyces cerevisiae strain is constructed with S. cerevisiae CEN PK2-1C, W303, FY1679, or BY4743 as a starting strain.

In the invention, the recombinant Saccharomyces cerevisiae strain is constructed through a method comprising the following steps in an arbitrary order:
S1: knock out the self-localization signal peptide of the lysophosphatidic acid acyltransferase coding gene, and connect the endoplasmic reticulum localization signal peptide at the C terminus to obtain the lysophosphatidic acid acyltransferase gene. Introducing the lysophosphatidic acid acyltransferase gene into Saccharomyces cerevisiae;
S2: knock out the own lysophosphatidic acid acyltransferase coding genes (SLC1, ALE1 and LOA1) of Saccharomyces cerevisiae;
S3: knock out the own triglyceride lipase coding genes (TGL3, TGL4, and TGL5) of Saccharomyces cerevisiae, to obtain the recombinant Saccharomyces cerevisiae strain.

Preferably, in Step S1, the lysophosphatidic acid acyltransferase coding gene is integrated into the genome of Saccharomyces cerevisiae by Cre/loxp technology, through a process comprising the following steps:
1) integrating and expressing the lysophosphatidic acid acyltransferase coding gene by the Cre/loxp system as shown in SEQ ID NO: 5, SEQ ID NO: 6, and SEQ ID NO:7, using constitutive promoters P_{TEF1}, P_{TDH1}, P_{PGK1}, P_{PYK}, P_{INO2}, P_{ITR1}, P_{ALD5}, P_{ION1}, P_{LEU2}, and P_{ZWF1}, and terminators T_{ADH1}, T_{DNM1}, T_{TPS1}, T_{TDH3}, T_{SLX5}, T_{ATP5}, and T_{CYC1}, and designing PCR primers so that the overlapping region of adjacent fragments of the gene expression frame reaches 40 to 100 bp, to construct a gene expression and integration frame of CrlPAAT1 (NCBI Reference Sequence: XP_042921325.1);
2) integrating the expression frame of the lysophosphatidic acid acyltransferase coding gene to the site 104c, 416d, 208c, 1622a, 308a, and 911b of Saccharomyces cerevisiae by the Cre/loxp method.

Preferably, in Step S2, the own lysophosphatidic acid acyltransferase coding genes ALE1, LOA1, and SLC1 of Saccharomyces cerevisiae are knocked out by the Cre/loxp method.

A second object of the present invention is to provide the use of the above-mentioned Saccharomyces cerevisiae strain in the preparation of human milk lipid substitute.

Preferably, the use includes the step of fermenting and producing human milk lipid substitutes by using glucose as a substrate.

Preferably, in the fermentation and production process, the pH is 6.0-8.0.

Preferably, in the fermentation and production process, the temperature is 20-30° C.

Preferably, the Saccharomyces cerevisiae strain is inoculated into a sterile medium with no amino nitrogen source, a YPD sterile medium with limited nitrogen source, an inorganic salt sterile medium, or a soybean peptone sterile medium with limited nitrogen source where glucose is used as a carbon source, and fermented with ventilation at pH 6.0-8.0, 200-300 rpm, and 20-30° C.

The present invention has the following beneficial effects.

With the recombinant Saccharomyces cerevisiae provided in the present invention, a human milk lipid substitute can be produced by fermentation in a sterile medium having glucose as a carbon source, which lays a foundation for the metabolic engineering of Saccharomyces cerevisiae to synthesize a human milk lipid substitute. The construction method for recombinant Saccharomyces cerevisiae provided in the present invention is simple and convenient and has a good application prospect.

### BRIEF DESCRIPTION OF THE DRAWINGS

FIG. 1 Change in localization of the heterologous gene CrlPAAT1;

FIG. 2 The triacylglycerol separated by thin-layer chromatography;

FIG. 3 Relative content of C16:0 in total fatty acids and Sn-2 position in different strains.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

Experimental materials involved:
250 ml shake flask, two-pan/single-pan analytical balance, Electric Sterilizer, recombinant strain, shaking table
Culture medium (g/L):
YNB medium: YNB 3.4, ammonium sulfate 10, glucose 20, uracil 0.05, histidine 0.05, leucine 0.05 and tryptophan 0.05;
YPD sterile medium with limited nitrogen source: glucose 20, yeast extract 10, and peptone 10;
Inorganic salt sterile medium: glucose 10, (NH₄)₂SO₄ 1, K₂HPO₄ 0.125, KHPO₄ 0.875, KI 0.0001, MgSO₄·7H₂O 0.5, CaCl₂·2H₂O 0.1, NaCl 0.1, stock solution of trace elements 1 mL, and stock solution of vitamins 1 mL;
Basal fermentation medium with limited nitrogen source: glucose 20, ammonium sulfate 4, KH₂PO₄ 2.5, and MgSO₄·7H₂O 0.5.
Seed medium: YNB medium, inorganic salt sterile medium, YPD sterile medium with a limited nitrogen source, basal fermentation medium with a limited nitrogen source.
Fermentation medium: YNB medium, inorganic salt sterile medium, YPD sterile medium with a limited nitrogen source, and basal fermentation medium with a limited nitrogen source.

Detection methods involved:
Gas chromatography-mass spectrometry (GC-MS): Shimadzu (GCMS-QP2010 SE), SH-Rtx-Wax column, mobile phase He, column temperature 100° C., injection volume 1 μL, split ratio 20:1, flow rate 1.0 ml/min.

In the present invention, recombinant S. cerevisiae CEN PK2-1C MATa; ura3-52; trp1-289; leu2-3,112; his3-Δ1; MAL2-8C; SUC2; S. cerevisiae W303 MATa; ura3-1; trp1-Δ1; leu2-3,112; his3-11; ade2-1; can1-100; S. cerevisiae FY1679MATa; ura3-52; trp1-Δ63; leu2-Δ1; his3-Δ200; GAL2 and S. cerevisiae BY4743 MATa; ura3-Δ0; met15-Δ0; leu2-Δ0; his3-Δ1; and lys-Δ0 are used as a starting strain, the lysophosphatidic acid acyltransferase coding gene derived from Chlamyolomonas is integrated into the genome of Saccharomyces cerevisiae by the Cre/loxp method, thereby obtaining a Saccharomyces cerevisiae strain capable of synthesizing a human milk lipid substitute (triacylglycerol).

Saccharomyces cerevisiae is also called baker's yeast or brewer's yeast. Saccharomyces cerevisiae is a yeast species most extensively related to human beings. As a safety strain for food, it has been used in making bread, steamed bread, other foods, and in the wine industry. In recent years, the scholars study the use of Saccharomyces cerevisiae to produce natural products, such as artemisinin and Panax notoginseng saponins. Saccharomyces cerevisiae has the advantages of high safety, low pathogenicity, high stress resistance and low probability of being contaminated by phage, so it also plays an important role in the field of genetic engineering. However, the triacylglycerol structure in Saccharomyces cerevisiae is quite different from that in human milk. Therefore, in order to obtain an engineering strain for the production of human milk lipid substitutes, the Cre/loxp technology is used to integrate an acyltransferase metabolic pathway into Saccharomyces cerevisiae, so as to achieve the effect of producing human milk lipid substitute.

Primers used in the following examples are shown in the table below:

| | |
|---|---|
| F1 | AGTATTATCTCTGCTGGTCGGTACTTAAATTGG |
| | (SEQ ID NO: 22) |
| R1 | AAAAAATCGGATGTTGAATGGGCATAAATATAAATGTATATATAAgctattacgccagctg |
| | (SEQ ID NO: 23) |
| F2 | TCATGACATTGACGAGAGTTTCTCTCGTACCCATATT |
| | (SEQ ID NO: 24) |
| R2 | TCAGGGGTAATTAACTTGTTAACTTTGGAGAAGTAAATGAAAAATG |
| | (SEQ ID NO: 25) |
| F3 | GAAAGATGGTAAACCCAACCATTTAGTCAAAACAGACATatagcttcaaaatgtttctactccttt |
| | (SEQ ID NO: 26) |
| R3 | CGAATACCTCAGAACATCATCTTTTCAGCAAGtagttctagaaaacttagattagattgct |
| | (SEQ ID NO: 27) |
| F4 | CATAAATATAAATGTATATATAAgctattacgccagctgaattggagcgacctcatgctatactgagaaagca |
| | (SEQ ID NO: 28) |
| R4 | CGAAGATGTTGTGGTTAGCGGCCATaattcttcgccagaggtttggtcaagtctc |
| | (SEQ ID NO: 29) |
| F5 | TGCTTGTTGTGCTGCTTGTTCTGACAAAAAAGATCTCAAAACAGAAGA |
| | (SEQ ID NO: 30) |
| R5 | aaaggagtagaaacattttgaagctatATGTCTGTTTTGACTAAATGGTTGGGTTTACCATCTTTCTT |
| | (SEQ ID NO: 31) |
| F6 | gcaatctaatctaagttttctagaactaCTTGCTGAAAAGATGATGTTCTGAGGTATTCG |
| | (SEQ ID NO: 32) |
| R6 | TGTTTTGTATGCTATTTCATTTTTCATTTACTTCTCCAAAGTTAACAAGTTAATTACCCCTGAAGCCGCCTTGC |
| | (SEQ ID NO: 33) |
| F7 | AACCAACAAACCAGGAACATGAGAAGAACCCAAAGTCATatagcttcaaaatgtttctactccttt |
| | (SEQ ID NO: 34) |
| R7 | aaaggagtagaaacattttgaagctatATGACTTTGGGTTCTTCTCATGTTCCTGGTTTGTTGGTT |
| | (SEQ ID NO: 35) |
| F8 | ttgaccaaacctctggcgaagaattATGGCCGCTAACCACAACATCTTCGCCGC |
| | (SEQ ID NO: 36) |
| R8 | TCTTCTGTTTTGAGATCTTTTTTGTCAGAACAAGCAGCACAACAAGCAGTT |
| | (SEQ ID NO: 37) |
| F9 | AAACCAGGAACATGAGAAGAACCCAAAGTATGGCCGCTAACCACAACATCTTCGCCGCT |
| | (SEQ ID NO: 38) |
| R9 | aaaggagtagaaacattttgaagctatATGTCAGAACAAGCAGCACAACAAGCAGTTAATAATGC |
| | (SEQ ID NO: 39) |
| F10 | TTACCTTCATATGACAAAATTTCGTTGGACTCATCGTC |
| | (SEQ ID NO: 40) |
| R10 | TACCTCAGAACATCATCTTTTCAGCAAGTATTTTTTTGCTCGAATTCACTGACGGAGGGA |
| | (SEQ ID NO: 41) |
| F11 | GCTTCAGGGGTAATTAACTTGTTAACTTCACATTTTTAGAGTAGTATATA |
| | (SEQ ID NO: 42) |
| R11 | CTTTAGCAATATCGGACAAACAGCCGTATTTTTTATTCT |
| | (SEQ ID NO: 43) |
| F12 | CTATCTTGGGGGTACGAAACTTAGCAATATATTTAGCAA |
| | (SEQ ID NO: 44) |
| R12 | TGGACCAGAACTACCTGTGAAATTAATGTTGGAAGCGATCAGCAGCAGAAT |
| | (SEQ ID NO: 45) |
| F13 | CTAACAAAATAGCAAATTTCGTCAAAAAATGGCCAAAAACGACAGATG |
| | (SEQ ID NO: 46) |
| R13 | GTGAAGACAAATTATCTAAGGTCCAACTCTACGAAGATTT |
| | (SEQ ID NO: 47) |
| F14 | ATAGACGTTTCTAAACCCAAATGAATCCAAGTTGCCGA |
| | (SEQ ID NO: 48) |
| F15 | TCAATGACGAGGTTCTCACCCCTGCCCAGGTATCTTGTATTTTGTCACCTCGTAGGAGCTACTT |
| | (SEQ ID NO: 49) |
| R14 | TCCTAGCGCTCACCAAGCTCTgAAATACTTATGAAGGCATGCATG |
| | (SEQ ID NO: 50) |
| R15 | AGGATCATTCTGGTAATACCCCATTTAAAATACCTCATCGAA |
| | (SEQ ID NO: 51) |
| F16 | GTTTCCCTCCGTCAGTGAATTCGAGCAAAAAAATACTTGCTGAAAAGATGATGTTCTGAGGTA |
| | (SEQ ID NO: 52) |
| R16 | TATATACTACTCTAAAAATGTGAAGTTAACAAGTTAATTACCCCTGAAGC |
| | (SEQ ID NO: 53) |
| F17 | TTCTGCTGCTGATCGCTTCCAACATTAATTTCACAGGTAGTTCTGGTCCAT |
| | (SEQ ID NO: 54) |
| R17 | ATCTGTCGTTTTTGGCCATTTTTTGACGAAATTTGCTATTTTGTTA |
| | (SEQ ID NO: 55) |
| F18 | CATGCATGCCTTCATAAGTATTTcAGAGCTTGGTGAGCGCTAGGAGTC |
| | (SEQ ID NO: 56) |
| R18 | CTACGAGGTGACAAAATACAAGATACCTGGGCAGGGGTGAGAACCTCGTCATTGA |
| | (SEQ ID NO: 57) |
| F19 | ATAGCCTCGACGCCAGCTTTGATTAGTTCATCCGGGTTCCCG |
| | (SEQ ID NO: 58) |
| F20 | CTTCAGGGGTAATTAACTTGTTAACTTTATCGTTTCCACTTTTTTCTGTCTTATTTTTTTTATTGATAG |
| | (SEQ ID NO: 59) |
| F25 | ACATCTTTGAGTTGCCGTTAAGCCTTGCTGAAAAGATGATGTTCTGAGGTATTCGTATCGCTAGCTTGATAC |
| | (SEQ ID NO: 60) |
| R19 | ATACCTCAGAACATCATCTTTTCAGCAAGGCTTAACGGCAACTCAAAGATGTGA |
| | (SEQ ID NO: 61) |
| R20 | TTGGTCCTATTCAGCGACCAATCAGTGCTTCCTGCCCACTTT |
| | (SEQ ID NO: 62) |
| R25 | AAAAGTGGAAACGATAAAGTTAACAAGTTAATTACCCCTGAAGCCGCCTTGCATG |
| | (SEQ ID NO: 63) |
| F21 | GGGATCCTCTTGCGAAGCACGCTCGCTGGGCCTGGAA |
| | (SEQ ID NO: 64) |
| F22 | ACAAAATAGCAAATTTCGTCAAAAAGAAAACACGGGCTTGCTTATATATCCTCTAGATATTCTT |
| | (SEQ ID NO: 65) |
| F26 | GAGTACAGGTATATGTAATAAAAGTCTGAATTAATTTCACAGGTAGTTCTGGTCCATTGGTGAAAGTTTGCGGCTTG |
| | (SEQ ID NO: 66) |
| R21 | GAACTACCTGTGAAATTAATTCAGACTTTTATTACATATACCTGTACTCCCTTCAATAATTA |
| | (SEQ ID NO: 67) |
| R22 | GTTATTATGTTGGCAATGGAAAAGTTGCGTACCGAGATACCGTAA |
| | (SEQ ID NO: 68) |
| R26 | ATATAAGCAAGCCCGTGTTTTCTTTTTGACGAAATTTGCTATTTTGTTAGAGTCTTTTACAC |
| | (SEQ ID NO: 69) |
| F23 | TGCTGGAGCAAGCTCCGTCTCCAGCTCGGGAAGTGT |
| | (SEQ ID NO: 70) |
| F24 | CGAGGTTCTCACCCCTGCCCAGGTATTGAACAATTCAGACATTTTTCAAAATTGAAATGAAAGACA |
| | (SEQ ID NO: 71) |
| F27 | ACAAGGAAGACGGTTCTGTTTCGTTGCTAGAGCTTGGTGAGCGCTAGGAGTCACTGCCAGGTATCGTTT |
| | (SEQ ID NO: 72) |
| R23 | CCTAGCGCTCACCAAGCTCTAGCAACGAAACAGAACCGTCTTCCTTGTGCTGTTTATGATG |
| | (SEQ ID NO: 73) |
| R24 | ATTTCCTATGAATAATCAAGACATAATGAATTTTCAATGGCTATTGAACTTCCC |
| | (SEQ ID NO: 74) |
| R27 | AATTTTGAAAAATGTCTGAATTGTTCAATACCTGGGCAGGGGTGAGAACCTCGTCATTGATGGACAGGT |
| | (SEQ ID NO: 75) |

### Example 1. Construction of Saccharomyces cerevisiae Strain (TG-1 to TG-4) Expressing Heterologous Acyltransferase

Based on the lysophosphatidic acid acyltransferase CrlPAAT1 (NCBI Reference Sequence: XP_042921325.1) derived from Chlamyolomonas publicized on NCBI, codon optimization was carried out according to the codon preference of Saccharomyces cerevisiae, and whole gene was synthesized. According to the design method of primers for overlap extension PCR, primers were designed to make the overlapping region of adjacent fragments of a gene expression frame reach 40-100 bp. Primers were designed to amplify the upstream and downstream homologous arms of the site 911b on the chromosome of Saccharomyces cerevisiae CEN PK2-1C, the promoters P_{TEF1}, P_{TDH1}, P_{PGK1}, P_{PYK}, P_{INO2}, P_{ITR1}, P_{ALD5}, P_{ION1}, P_{LEU2} and P_{ZWF1}, the terminators T_{ADH1}, T_{DNM1}, T_{TPS1}, T_{TDH3}, T_{SLX5}, T_{ATP5} and T_{CYC1}, and the signal peptide (HDEL) fragment.

Using the whole-gene synthesized CrlPAAT1 plasmid as a template, primers were designed to amplify heterologous lysophosphatidic acid acyltransferase. Using the plasmid pMHyLp-LEU as a template (as shown in SEQ ID NO:5), a defective tag fragment was obtained by PCR amplification, and a gene integration frame was obtained by overlap extension PCR.
(1) Using the primers F1 and R1, and F2 and R2, the upstream and downstream homologous arm fragments of the site 911b were amplified; using the primers F3 and R3, the promoter P_{TEF1} fragment was obtained by amplification; using the primers F4 and R4, the terminator T_{ADH1} fragment was obtained by amplification; using the primers F5 and R5, the CrlPAAT1 gene fragment as shown in SEQ ID NO: 4 was obtained by amplification; and the pMHyLp-LEU fragment was amplified using the primers F6 and R6. A gene expression frame was constructed by overlap extension PCR and transferred into Saccharomyces cerevisiae CEN PK2-1C, to obtain the strain TG-1.
(2) Using F5 and R7, the self-localization signal peptide (as shown in SEQ ID NO: 8) of the CrlPAAT1 gene (having a gene sequence as shown in SEQ ID NO: 4) was knocked out, to obtain the mCrlPAAT1 gene. A gene expression frame constructed with the homologous arms in (1), the (F7 and R3) promoter, the terminator, and the defective tag fragment following the process in (1) was transferred into Saccharomyces cerevisiae CEN PK2-1C, to obtain the strain TG-2.
(3) Using the primers F8 and R8, and F9 and R9, the fragments of signal peptide HDEL (as shown in SEQ ID NO: 13) connected to the C and N terminals of mCrlPAAT1 gene were obtained by amplification. A gene expression frame constructed by the fragment of signal peptide HDEL connected to the C terminal of mCrlPAAT1 gene and the fragment of mCrlPAAT1 gene in (2) was transferred to Saccharomyces cerevisiae CEN PK2-1C to obtain the strain TG-3. A gene expression frame constructed by the fragment of signal peptide HDEL connected to the N terminal of mCrlPAAT1 gene and the fragment of mCrlPAAT1 gene in (2) was transferred to Saccharomyces cerevisiae CEN PK2-1C to obtain the strain TG-4.

The strains TG-3 and TG-4 were detected, and it was found that the relative content of C16:0 at the Sn-2 position of TAG in strain TG-3 was increased to 40%, so the strain TG-3 was used for subsequent experiments. In the above process, the constructed gene integration frame was transformed into competent cells of Saccharomyces cerevisiae by lithium acetate transformation. Colonies were picked up for PCR verification and some transformants verified to be correct by PCR were used for sequencing verification.

### Example 2. Construction of Saccharomyces cerevisiae Strain (TG-5-TG-14) Highly Producing Human Milk Lipid Substitute

According to the design method of primers for overlap extension PCR, using the genome of Saccharomyces cerevisiae CEN PK2-1C as a template, the upstream and downstream homologous arms of SLC1 (F10, R10 and F11, R11), ALE1 (F12, R12 and F13, R13) and LOA1 gene (F14, R14 and F15, R15) were obtained by amplification. Using the plasmid pMHyLp-LEU (F16, R16), pMHyLp-HIS (F17, R17) and pMHyLp-TRP (F18, R18) as a template (as shown in SEQ ID NO: 5 to SEQ ID NO: 7), to obtain defective expression frame fragments by amplification. A gene integration frame was obtained by overlap extension PCR. On the basis of strain TG-3, single knockout, double knockout and triple knockout of the above three genes were performed respectively, to obtain strains TG-5 (SLC1 knock-out), TG-6 (ALE1 knock-out), TG-7 (LOA1 knock-out), TG-8 (SLC1 and ALE1 knock-out), TG-9 (SLC1 and LOA1 knock-out), TG-10 (ALE1 and LOA1 knock-out) and TG-11 (SLC1, ALE1 and LOA1 knock-out).

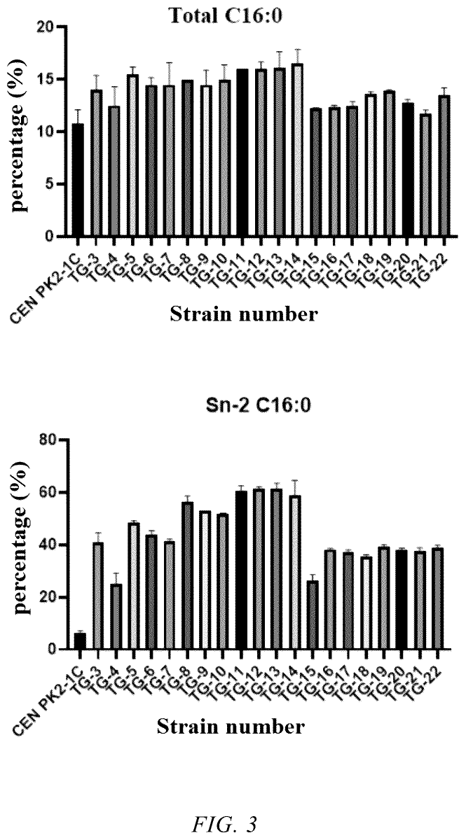

The relative content of C16:0 at total fatty acids and Sn-2 position of strains TG-5 to TG-11 were detected. The results are shown in FIG. 3. The C16:0 content in total fatty acids of strain TG-11 is the highest, and the relative content of C16:0 at Sn-2 is up to 60% or more.

On the basis of strain TG-11, the genes related to the hydrolysis pathway of triacylglycerol in Saccharomyces cerevisiae were knocked out. The primers were designed following the above method. After amplification, the upstream and downstream homologous arms of TGL3 (F19, R19 and F20, R20), TGL4 (F21, R21 and F22, R22) and TGL5(F23, R23 and F24, R24) genes and defective expression frame fragments (F25-F27 and R25-R27) were obtained. These three gene knock-out expression frames were transferred into strain TG-11 to obtain strains TG-12 (TGL3 knock-out), TG-13 (TGL3 and TGL4 knock-out) and TG-14 (TGL3, TGL4, and TGL5 knock-out).

The relative content of C16:0 at total fatty acids and the Sn-2 position of strains TG-12 to TG-14 were detected. The results are shown in FIG. 3. The final detection results show that the total fatty acid content of strain TG-14 is slightly increased compared with TG-11, and the relative content of C16:0 at the Sn-2 position of TAG is increased to 60%. In the above process, the constructed gene integration frame was transformed into competent cells of Saccharomyces cerevisiae by lithium acetate transformation. Colonies were picked up for PCR verification and some transformants verified to be correct by PCR were used for sequencing verification.

### Example 3. Production of Human Milk Lipid Substitute Through Fermentation by Recombinant Saccharomyces cerevisiae

In the invention, all recombinant Saccharomyces cerevisiae strains were used for fermentation through the following process. The recombinant Saccharomyces cerevisiae strain was streaked with amino-free nitrogen source plates (lacking the amino acid corresponding to the deficient type), and cultured at 30° C. until a large number of colonies were grown.

A single colony was picked to a seed culture medium (any culture medium selected from a sterile medium with no amino nitrogen source, a YPD sterile medium with limited nitrogen source, an inorganic salt sterile medium, and a soybean peptone sterile medium with limited nitrogen source where glucose is used as a carbon source is used as a fermentation medium), cultured at 30° C. and 220 rpm for 18-20 h to the logarithmic phase of cell growth.

The seed culture was inoculated into the fermentation medium (any culture medium selected from a sterile medium with no amino nitrogen source, a YPD sterile medium with limited nitrogen source, an inorganic salt sterile medium, and a soybean peptone sterile medium with limited nitrogen source where glucose is used as a carbon source is used as a fermentation medium) according to an initial inoculation amount of 2-5%, cultured at 30° C. and 220 rpm for 72 h. After 72 h, the culture was stopped, and the yeast cells after fermentation were centrifuged, and freeze-dried for subsequent detection and analysis.

### Example 4. Extraction and Detection of Human Milk Lipid Substitute Produced by Recombinant Saccharomyces cerevisiae

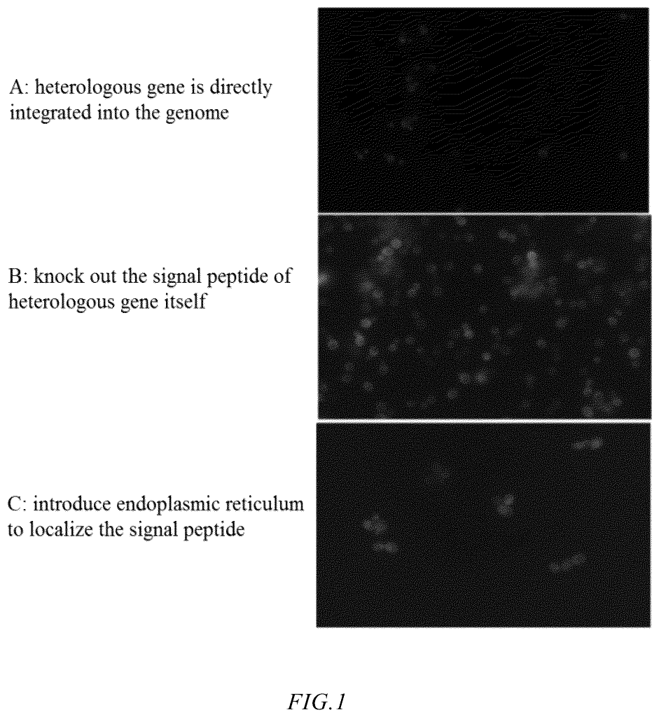

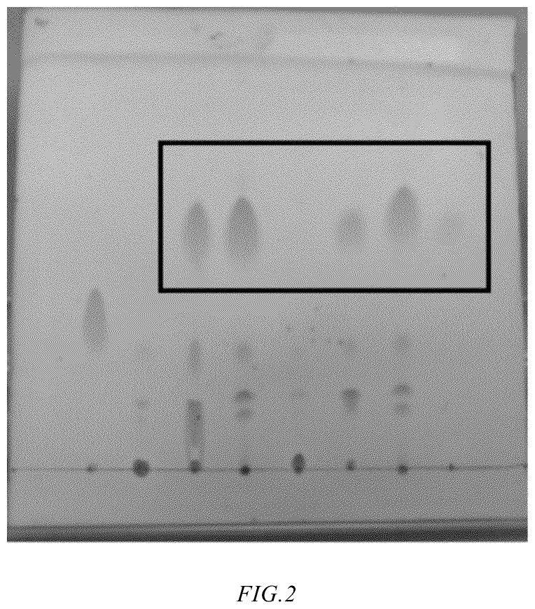

In the invention, the lipid in all the recombinant Saccharomyces cerevisiae strains were extracted through the following process. Tripentadecanoin was added to freeze-dried cells as an internal standard. Methanol and glass beads were added for shaking and crushing the cells, and then the crushed solution was transferred to a new volumetric flask. Then, added the Chloroform, the solution was ultrasonicated in a water bath for 10 min. The supernatant after ultrasonic treatment was transferred to a new volumetric flask, 1.5 ml of methanol/chloroform solvent was added again for extraction. This step was repeated twice. The obtained lipid extracts were combined, and 2.5 ml of chloroform and 3 ml of NaCl aqueous solution were added to the combined solution. The sample was shaken vigorously, and centrifuged at 10000 rpm for 5 min. The upper liquid was discarded, and the lower organic phase was transferred to a new glass tube. The organic phase was blown to dryness with a nitrogen blower and then redissolved in n-hexane. Thin-layer chromatography was used to separate the extracted lipid. The developing agent was n-hexane:ether:acetic acid (70:30:1, v:v:v). Bromothymol blue was used to stain the lipid on the chromatographic plate. According to the staining results, the triglyceride band was taken by a sampler. Then the separated triglyceride sample was transferred to a 10 ml centrifuge tube, and 0.2 ml of n-hexane was added to the test tube. Then 50 mg of pancreatic triglyceride lipase and 2 ml of Tris-HCL (pH=8) buffer were added and shaken carefully. Then 0.5 ml of sodium cholate solution and 0.2 ml of calcium chloride solution were added. The centrifuge tube was capped and shaken carefully. Then the centrifuge tube was allowed to stand in a water bath at 40° C. for 5 min, and the centrifuge tube was shaken by hand. After standing in the water bath, the centrifuge tube was vibrated on a vibrator for 2 min. Then 1 ml of hydrochloric acid solution (6 mol/L) and 1 mL of diethyl ether were added. The centrifuge tube was capped and shaken vigorously for 10 s. After centrifugation at 4000 rpm for 4 min, the organic phase was pipetted, blown with nitrogen to 200 μL, and then determined by GC-MS. The gene localization results are shown in FIG. 1, the results of thin-layer chromatography are shown in FIG. 2, and the C16:0 content is shown in FIG. 3.

As can be seen from FIG. 1, when the heterologous gene CrlPAAT1 was directly integrated into the genome of Saccharomyces cerevisiae, the expression level of the gene was low. The expression of the mCrlPAAT1 gene, obtained by knocking out the signal peptide of the heterologous gene itself, is significantly increased in Saccharomyces cerevisiae, and it is found that the gene is mainly expressed freely in the cytoplasm. The rmCrlPAAT1 gene is obtained after a signal peptide is added to the C-terminal of the mCrlPAAT1 gene, and is highly expressed in Saccharomyces cerevisiae and localized to the endoplasmic reticulum.

### Comparative Example 1

The lysophosphatidic acid acyl transferase was replaced by the following sequence (1), its own localization signal peptide (having a sequence as shown in SEQ ID NO: 21) was knocked out, and the signal peptide HDEL was added to the C terminal of the obtained gene following specific steps as described in Example 1 to obtain strain TG-15.

The lysophosphatidic acid acyl transferase was replaced by the following sequence (2), and the signal peptide HDEL was added to the C terminal of the obtained gene following specific steps as described in Example 1 to obtain strains TG-16 and TG-17, respectively.
(1) Brassica-derived lysophosphatidic acid acyltransferase LPAT1 (NCBI Reference Sequence: AF111161), the gene sequence as shown in SEQ ID NO: 1;
(2) Homo sapiens-derived lysophosphatidic acid acyltransferase AGPAT1 (NCBI Reference Sequence: NC_000006.12) and AGPAT2 (NCBI Reference Sequence: CAH71722.1), the gene sequence as shown in SEQ ID NO: 2 and SEQ ID NO: 3, respectively.

### Comparative Example 2

Replace the signal peptide HDEL with the following signal peptide: (1) SRP14 (having a gene sequence as shown in SEQ ID NO: 9), (2) SRP54 (having a gene sequence as shown in SEQ ID NO: 10), (3) CYB5 (having a gene sequence as shown in SEQ ID NO: 11), (4) SEC 12 (having a gene sequence as shown in SEQ ID NO: 12), or (5) FEHDEL (having a gene sequence as shown in SEQ ID NO: 14), following specific steps as described for strain TG-3 in Example 1, to obtain strains TG-18 to TG-22 respectively.

Apparently, the above-described embodiments are merely examples provided for clarity of description and are not intended to limit the implementations of the present invention. For those of ordinary skill in the art, on the basis of the above description, other changes or changes in various forms can also be made. It is not necessary and impossible to exhaustively list all the implementation manners here. The obvious changes or modifications derived therefrom are still within the protection scope of the present invention.

1. A Saccharomyces cerevisiae strain for producing a human milk lipid substitute, wherein in the Saccharomyces cerevisiae strain, a lysophosphatidic acid acyltransferase CrlPAAT1 is expressed, SLC1, ALE1 and LOA1 genes encoding lysophosphatidic acid acyltransferase are knocked out, and TGL3, TGL4 and TGL5 genes encoding triglyceride lipase are knocked out; and in the lysophosphatidic acid acyltransferase CrlPAAT1, a self-localization signal peptide is knocked out, and an endoplasmic reticulum localization signal peptide is connected to the C terminal.

2. The Saccharomyces cerevisiae strain according to claim 1, wherein the lysophosphatidic acid acyltransferase CrlPAAT1 has a nucleotide sequence as shown in SEQ ID NO: 4.

3. The Saccharomyces cerevisiae strain according to claim 1, wherein the self-localization signal peptide of the lysophosphatidic acid acyltransferase CrlPAAT1 has a nucleotide sequence as shown in SEQ ID NO: 8.

4. The Saccharomyces cerevisiae strain according to claim 1, wherein the endoplasmic reticulum localization signal peptide has a nucleotide sequence as shown in SEQ ID NO: 13.

5. The Saccharomyces cerevisiae strain according to claim 1, wherein plasmid pMHyLp-LEU is used as an expression vector for the gene encoding the lysophosphatidic acid acyltransferase CrlPAAT1.

6. The Saccharomyces cerevisiae strain according to claim 1, wherein SLC1 has a nucleotide sequence as shown in SEQ ID NO: 15, ALE1 has a nucleotide sequence as shown in SEQ ID NO: 16, and LOA1 has a nucleotide sequence as shown in SEQ ID NO: 17.

7. The Saccharomyces cerevisiae strain according to claim 1, wherein TGL3 has a nucleotide sequence as shown in SEQ ID NO: 18, TGL4 has a nucleotide sequence as shown in SEQ ID NO: 19, and TGL5 has a nucleotide sequence as shown in SEQ ID NO: 20.

8. The Saccharomyces cerevisiae strain according to claim 1, wherein the Saccharomyces cerevisiae strain is constructed with Saccharomyces cerevisiae CEN PK2-1C, W303, FY1679 or BY4743 as a starting strain.

9. Use of the Saccharomyces cerevisiae strain according to claim 1 in the preparation of a human milk lipid substitute.

10. The use according to claim 9, wherein glucose is used as a substrate to produce a human milk lipid substitute by fermentation.
